(19) United States
(12) Patent Application Publication
(10) Pub. No.: US 2024/0058518 A1
(43) Pub. Date: Feb. 22, 2024

(54) FILTER FOR REMOVING SUBSTANCES FROM BLOOD

(71) Applicant: Fresenius Hemocare Italia S.R.L., Mirandola (IT)

(72) Inventors: Paolo Bonaguidi, Carpi (IT); Serena Borghi, Mirandola (IT); Nicoletta Sala, Carpi (IT)

(21) Appl. No.: 18/250,379

(22) PCT Filed: Nov. 10, 2020
(86) PCT No.: PCT/EP2020/081612

§ 371 Date: Apr. 25, 2023

(51) Int. Cl. A61M 1/36; B01D 39/16

(52) U.S. Cl. CPC A61M 1/3633; B01D 39/1623; B01D 2239/0492; B01D 2239/0618

(57) ABSTRACT

Blood filters for enhanced leukoreduction with platelet recovery are disclosed. The filters include a non-woven, porous filter coated with an acetonic copolymer solution.

### FIELD OF THE DISCLOSURE

The present disclosure generally relates to filters for removing substances such as selected components and/or impurities from a biological fluid such as blood. More particularly, the present disclosure is directed to blood filters that effectively remove undesirable components such as leukocytes while allowing for effective recovery of desirable blood components such as platelets. Even more particularly, the present disclosure is directed to blood filter assemblies that include such filters and to methods for making such filters and filter assemblies.

### BACKGROUND

Using various manual and automated systems and methods, whole blood is collected and often separated into its clinical components (typically red blood cells, platelets, and plasma). The whole blood or the collected components are typically individually stored and used to treat a variety of specific conditions and diseased states.

Before transfusing the blood or collected blood components (“blood product”) to a recipient in need of the components, it is often desirable to minimize the presence of impurities or other materials that may cause undesired side effects in the recipient. For example, because of possible reactions, it is generally considered desirable to reduce the number of leukocytes in the blood product before storage, or at least before transfusion (i.e., “leukoreduction”).

Filters are widely used to accomplish leukoreduction in blood products today (e.g., warm and cold filtration of leukocytes from whole blood, red cells, and/or platelet products). Filters typically include a filter media disposed between mating walls of a filter housing. Inlet and outlet ports associated with the housing provide flow paths to and from the interior of the filter. The walls of the housing may be made of a rigid, typically plastic, material, or of flexible material (typically PVC). Due to the importance of filtering blood or blood components, there exists an ongoing desire to improve the construction and performance of biological fluid filters.

### SUMMARY

In one aspect the present disclosure is directed to a filter medium for removing substances from blood. The filter medium includes a porous, polymeric non-woven material for removing selected components from blood and recovering other selected components. The filter medium includes a coating applied to the porous, polymeric material.

In another aspect, the present disclosure is directed to a blood filter assembly including a housing with an inlet and an outlet, a pre-filter, a post-filter and a filter medium located between the pre-filter and the post filter wherein the filter medium includes one or more sheets of the filter material described above.

In a further aspect, the present disclosure is directed to a method of making a filter for removing substances from blood. The method includes forming a fabric sheet made of a material that includes a polyether-ester copolymer, and coating at least one side of the fabric sheet with a coating solution that is a copolymer of vinyl acetate and vinyl pyrrolidone.

### BRIEF DESCRIPTION OF DRAWINGS

FIG. 1 is a front view of a representative filter assembly in accordance with the present disclosure;

FIG. 2 is a side, cross-sectional view taken along 2-2 of the filter assembly of FIG. 1; and

FIG. 3 is an exploded view of a filter assembly of the present disclosure.

### DETAILED DESCRIPTION OF THE EMBODIMENTS

The present disclosure is generally directed to filters for the removal of selected components from blood. The term “blood” includes whole blood and blood components, such as red blood cells, that have been separated from whole blood. The filters described herein are particularly well-suited (but not limited to) the filtration of whole blood.

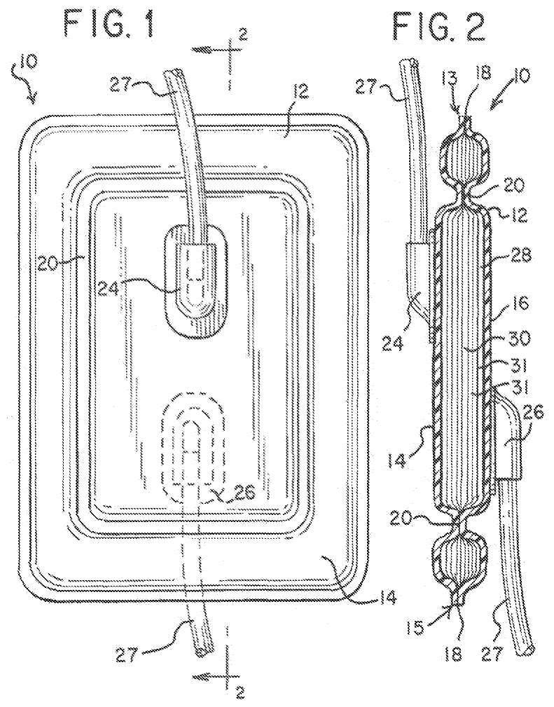

FIG. 1 shows one embodiment of a filter assembly in accordance with the present disclosure. Filter 10 is suited for removal of selected components and/or impurities from a biological fluid such as blood. Filter assembly 10 includes housing 12, which includes outer walls 14 and 16 (FIG. 2). Housing 12 and, indeed, filter 10 are preferably made of a biocompatible material that may also be sterilized using conventional sterilization techniques commonly used in the assembly of disposable blood processing sets such as autoclaving, gamma-ray and/or electron-beam. In one embodiment, housing walls 14, 16 may be made of a rigid, polymeric material sealed at or near the periphery thereof. The sealing of walls 14, 16 may be achieved by adhesive, welding or other forms of sealing attachment.

In a preferred embodiment, as shown in FIGS. 1 and 2, housing walls 14 and 16 may be made of a soft, flexible polymeric material. Examples of suitable polymeric materials for housing walls 14 and 16 include polyvinyl chloride and/or polyolefin. As shown in FIGS. 1 and 2, housing walls 14 and 16 may be joined along their peripheral edges to form a seal 18. In the embodiment of FIGS. 1 and 2, an additional inner peripheral seal 20 may also be provided, as described in U.S. Patent Application Publication US 2002/0113003, the contents of which are incorporated herein by reference. Seals 18 and 20 define a cushioned peripheral portion.

Typically, filter assemblies of the type described herein may be included as part of a disposable fluid processing set or kit where, in its most basic form, the biological fluid is introduced from a connected or pre-connected source, passed through the filter 10 and collected in a pre-attached container after it has passed through the membrane and the undesirable components captured by the filter medium. Thus, walls 14 and 16 may include inlet and outlet ports 24 and 26, respectively, to allow for introduction and exit of the fluid. Ports 24 and 26 communicate with an internal chamber, defined by housing walls 14 and 16. Ports 24 and 26 may be carried by walls 14 and 16, as shown in FIGS. 1 and 2. Ports 24 and 26 may be separately attached to housing walls 14, 16 or integrally molded with housing walls 14 and 16. As shown in FIGS. 1 and 2, the inlet and outlet ports 24 and 26 may be located in a diametrically opposed relationship on walls 14 and 16. Thus, for example, inlet port 24 may be positioned closer to the “top” peripheral edge 13 of filter 10 on wall 14, whereas outlet port 26 may be positioned closer to the “bottom” peripheral edge 15 of filter 10 and wall 16. Of course, it will be appreciated that the relative locations of ports 14 and 16 may be otherwise modified or provided. Ports 24 and 26 define internal flow paths which establish fluid communication between the interior chamber and tubing 27 leading to other containers or parts of a disposable processing set in which filter 10 is included.

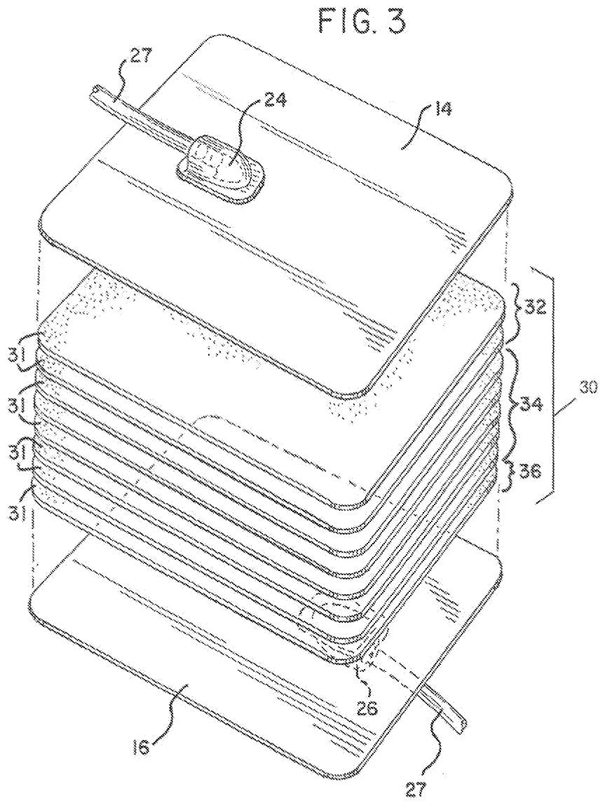

As shown in FIGS. 2-3, housing walls 14 and 16 accommodate a filter medium 30. In one embodiment, filter 30 may be provided as a pad that includes a plurality of stacked sheets with pores sized to prevent passage of leukocytes while allowing other desirable blood components, such as erythrocytes and platelets to pass. In one embodiment, as shown in FIG. 2, filter 30 may include a plurality of sheets 31 wherein each sheet 31 includes pores of a desired diameter and/or size and distribution. In one embodiment, sheets 31 may be made of melt blown, non-woven fibers. In accordance with the present disclosure, the fibers may be made of a suitable polymeric material, described in greater detail below.

As shown in FIG. 3, filter 30 may be made of a plurality of melt blown, non-woven fiber sheets. In addition, groups of sheets may provide a filter medium with filter portions selected to perform particular functions. For example, filter 30 may include a filter portion made up of a plurality of sheets wherein the filter portion 32 and/or the sheets that make up portion 32 adjacent or closest to housing wall 14 and inlet port 24 has/have a selected porosity that provides for the removal of microaggregates and smaller sized particulates. Although FIG. 3 shows two sheets (for representative purposes only), portion 32 may include one or more sheets and may typically include, but is not limited to, 1 to 5 sheets to provide a “pre-filter.”

Filter medium 34 may provide the primary or main filter and may likewise include a plurality of sheets of selected porosity. Although only 5 sheets are shown (for representative purposes only), the number of sheets that make up the primary or main filter may be anywhere from 10 to 40, wherein each sheet has a thickness of approximately 10 μm to 500 μm. In one embodiment, approximately 30 sheets may be included in filter medium 34.

Filter portion 36 may provide for the filtration of additional components and/or serve as a spacer element between filter medium 34 and housing wall 16. Filter portion 36 which may also include a plurality of sheets (although only 1 sheet is shown for representative purposes only) is positioned downstream of filter medium 34 closer to housing wall 16 and outlet port 26 and may be referred to as a “post-filter.” As seen in FIG. 2, filter portions 32, 34 and 36 may be brought together and sealed together to provide a unitary filter pad. Alternatively, some or all of the individual sheets of each of the filter portions 32, 34 and 36 may be brought together and sealed at an inner seal 20 with housing walls 14 and 16, as shown in FIG. 2.

Filter medium 34 may be a non-woven, material made from melt blown fibers as described in U.S. Pat. No. 7,736,516, the contents of which is incorporated herein by reference in its entirety. As described therein, each sheet of filter medium 34 may be made of a suitable polymeric material such as a polyether-ester copolymer (PEC).

The polyether-ester copolymer may be obtained by polycondensation in a melt of at least one alkyleneglycol, at least one aromatic dicarboxylic acid or an ester thereof and a polyalkylene oxide glycol. The alkyleneglycol may contain 2-4 carbon atoms and the preferred glycol is butyleneglycol.

Suitable aromatic dicarboxylic acids include terephthalic acid, 1,4-naphtalenedicarboxylic acid and 4,4′-diphenyldicarboxylic acid.

Preferred polyalkylene oxide glycols include polybutylene oxide glycol, polypropylene oxide glycol and polyethylene oxide glycol or combinations thereof; particularly preferred is a block copolymer of polypropylene oxide (PPO)/polyethylene oxide (PEO).

The resulting polymer has a backbone built up of a hard segment (hydrophobic) of repeating units, derived from the alkyleneglycol (preferably 1,4-butandiol) and the aromatic dicarboxylic acid (preferably terephthalic acid or dimethylterephthalate) and a soft hydrophilic segment derived from polyalkylene oxide glycol.

A preferred resulting polymer structure is:

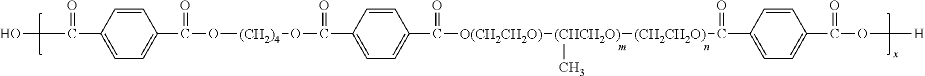

Suitable polyalkylene oxide glycols are commercially available, such as e.g. Pluronic PE6002.TM., which is polypropylene oxide end capped with ethylene oxide glycol from Basf (ethylene oxide:propylene oxide=36/64-weight ratio).

The copolyether esters described herein are commercially available or can be prepared according to known polycondensation processes, preferably according to the process described in U.S. Pat. No. 6,441,125, incorporated herein by reference, by polycondensation in the melt of the above-mentioned components in the presence of a catalyst based on a combination of titanium and a bivalent metal in a single compound or a combination of titanium and a bivalent metal containing compounds, wherein the molecular ratio of titanium to the bivalent metal is preferably lower than 1.5.

The bivalent metals are preferably alkaline earth metals, preferably magnesium; titanium is preferably in the form of a metal organic compound, such as titanium alkoxide or a titanium ester.

In accordance with the present disclosure, the copolyether-ester herein described can be processed by melt-blowing to obtain fibers having a diameter in the range of less than 6 μm and preferably less than 3 μm; the preferred mean diameter being in the range from 1.8 to 2.2 μm.

The amount of the polyalkylene oxide glycol in the copolyether ester is preferably in the range of from 0.1 to 20% by wt. or 0.03-6% by wt. of polyethylene oxide.

Leukoreduction and/or platelet recovery with the filter media may be further enhanced by coating the surface(s) of the filter medium 34 (or each sheet thereof) with a coating solution. The coating solution may be a polymer obtained by the polyreaction of a hydrophobic and hydrophilic monomer.

Examples of suitable hydrophobic monomers that may be used in the coating solution include vinyl ester such as vinyl acetate; alkenes such as ethylene, propylene, hexane-1, heptene-1, vinyl cyclohexane, 3,3-dimethyl-1-propene, 3-methyl-1-diisobutylene, 4-methyl pentene-1; and alkyl(meth)acrylates which are derived from saturated alcohols, for example methyl acrylate, ethyl(meth)acrylate, propyl(meth)acrylate, n-butyl(meth)acrylate, tert.-butyl(meth)acrylate, the expression (meth)acrylates incorporating methacrylates and acrylates, as well as mixtures of both.

Examples of suitable hydrophilic monomers include monomers with an acid group, for example vinylphosphonic acid, vinyl sulphonic acid, acrylic acid and methacrylic acid; monomers with a basic group, in particular vinyl pyridine, 3-vinyl pyridine, 2-methyl-5-vinyl pyridine, 3-ethyl-4-vinyl pyridine, 2,3-dimethyl-5-vinyl pyridine, N-vinyl pyrrolidone, 2-vinyl pyrrolidone, N-vinyl pyrrolidine and 3-vinyl pyrrolidine; as well as monomers with a polar group, such as 2-methacryl oxyethylphosphorylcholin, vinyl alcohol; vinyl alcohol can be obtained after polymerisation by saponifying vinyl esters.

More particular examples of suitable coating polymers/copolymers include, but are not limited to, N-vinyl-2-pyrrolidone (NVP) with vinyl acetate (VAc), 2-hydroxyethyl methacrylate (HEMA) with vinyl acetate (VAc), N-vinyl-2-pyrrolidone with 2-ethoxyethyl methacrylates (2-EMMA), N-vinyl-2-pyrrolidone (NVP) with methyl methacrylate (MMA) and vinyl alcohol (VA) with vinyl acetate (VAc).

In one embodiment, the coating solution may be an acetonic solution of a selected polymer or copolymer. In a more particular embodiment, the coating solution may be a solution of vinyl acetate and vinyl pyrrolidone (VA/VP) statistical copolymer. The weight ratio of vinyl acetate to vinyl pyrrolidone may be between 10:1 and 4:1, more preferably between 9:1 and 5:1 and even more preferably between 8:1 and 6:1. In one embodiment, the weight ratio of vinyl acetate and vinyl pyrrolidone (VA/VP) may be 7:1. For example, the coating solution may include 87.5% wt vinyl acetate and 12.5% wt vinyl pyrrolidone. Other copolymer solutions may also be used to coat filter medium 30 and enhance one or both of leukoreduction and platelet recovery. Additional properties of the coating solution described above are described in U.S. Pat. No. 7,775,376, the contents of which are incorporated herein by reference.

A sheet of the non-woven filter medium may be immersed in a bath of the acetonic coating solution for a period of time, sufficient to coat both surfaces of the filter medium 34. The coating solution preferably includes approximately 1-5% polymer and more preferably is a 1-3% polymeric solution or more preferably 2% (95%-99% wt acetone, and more preferably 97-99% or more preferably 98% wt acetone). After immersion, excess coating solution is allowed to drip off the surfaces of the medium. Once excess coating solution has been removed, filter medium is dried for a selected period of time and/or at a selected drying temperature. Drying may be carried out in an oven or by using a drying drum in ways that will be recognized by those of skill in the art. In one embodiment, filter medium may be dried at a temperature of between 54°-111° C. for a period of approximately 3-7 seconds and preferably approximately 5 seconds.

After the roll of coated filter medium (fabric) has been dried, the roll may be cut into individual sheets 31. A plurality of these individual sheets may be stacked and combined with sheets of a pre-filter and/or post-filter to arrive at a filter pad for inclusion in a filter assembly as shown in FIG. 1.

Coated filters made in accordance with the method described above further enhance leukoreduction and platelet recovery. Set forth below are test results that demonstrate the improved leukoreduction and platelet recovery properties of the coated filters described herein.

### EXAMPLE

Small-scale filters (soft housing, square shape, surface area 15 cm²) were produced with 20 layers of fabric (20 layers of filter medium, and 3 layers of a pre-filter and one layer of a post-filter wherein the pre-filter included one layer of spunbond PET and two layers of non-woven, meltblown PBT and the post filter comprised one layer of the spunbond PET.) Two different filter media, “control” and “PEC-sample” as described below, were compared. The control was produced with standard PBT combined with VAVP coating polymer (VA/VP weight ratio of 7, 0.22% w/w acetonic solution) while the PEC-sample included the PEC fabric combined with VAVP coating polymer (VA/VP weight ratio of 7, 2% w/w acetonic solution).

Blood units were collected from informed donors in PVC bags with 70 ml of CPD (500 ml±10%, CPD excluded). After collection, the blood bags were stored at room temperature on butane-1,4-diol cooling plates up to 24 hours. Before filtration, collected blood was connected to the filter system. Three filtrations were performed for each bag (160 g collect blood for each filter). Filtration was performed by gravity.

Samples were collected before and after filtrations from blood bags in order to evaluate white blood cell (WBC) removal and platelet (PLT) recovery (cell counts by Sysmex KX-21 N automated hematology analyzer). In the filtered blood, WBCs were counted by means of flow cytometry. The total filtration time at 160 g blood collection was also measured. Results are set forth in Table 1 below.

TABLE 1

| | Blood temperature | Filtration time | residual WBC/ | residual PLT/ | PLT recovery |
|---|---|---|---|---|---|
| Filter type | before filtration (° C.) | (hh:mm:ss) | unit ×10E6 | unit ×10E9 | (%) |
| Control Mean | 21.6 | 00:26:37 | 0.09 | 1.3 | 5.0 |
| St. Dev. | 0.4 | 00:04:58 | 0.05 | 1.9 | 7.5 |
| PEC Sample Mean | 21.8 | 00:17:11 | 1.11 | 24.0 | 74.7 |
| St. Dev. | 0.7 | 00:01:51 | 0.8 | 5.0 | 9.5 |

As shown in Table 1, the use of the coated PEC as fabric material increases the ability to recover platelets while allowing for adequate leukocyte removal and shortens filtration time when compared with other fabrics coated with the coating solution described herein.

It will be understood that the embodiments described above are illustrative of some of the applications of the principles of the present subject matter. Numerous modifications may be made by those skilled in the art without departing from the spirit and scope of the claimed subject matter, including those combinations of features that are individually disclosed or claimed herein. For these reasons, the scope hereof is not limited to the above description but is set forth in the following claims.

1. A filter medium for removing substances from blood comprising:
a porous, polymeric non-woven fabric for removing selected components from blood and recovering other selected components, said fabric comprising a non-woven material having hydrophilic and hydrophobic segments; and
a coating applied to said porous, polymeric fabric.

2. The filter medium of claim 1 wherein said porous, polymeric fabric comprises a polyether-ester copolymer.

3. The filter medium of claim 1, wherein said porous, polymeric fabric comprises a polymeric structure of:

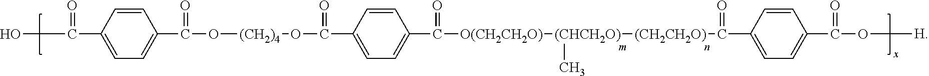

4. The filter medium of claim 1, wherein said hydrophobic segments include repeating units derived from an alkylene glycol and at least one aromatic dicarboxylic acid or ester.

5. The filter medium of claim 1, wherein said hydrophilic segments derived from at least one polyalkylene oxide glycol.

6. The filter medium of claim 1, wherein said coating is obtained by the polyreaction of a hydrophobic and hydrophilic monomer.

7. The filter medium of claim 6 wherein said hydrophobic monomer is selected from the group consisting of vinyl esters, alkenes and alkyl methacrylates and the hydrophilic monomers are selected from the group consisting of monomers with an acid group, monomers with a basic group and monomers with a polar group.

8. The filter medium of claim 1, wherein said coating comprises an acetonic solution comprising a copolymer of vinyl acetate and vinyl pyrrolidone.

9. The filter medium of claim 8 wherein the ratio of vinyl acetate to vinyl pyrrolidone is between 10:1 to 4:1.

10. A blood filter assembly comprising a housing including an inlet and an outlet;
a pre-filter;
a post-filter; and
a filter medium located between said pre-filter and said post filter said filter medium comprising one or more sheets of the filter medium, wherein the filter medium comprises:
a porous, polymeric non-woven fabric for removing selected components from blood and recovering other selected components, said fabric comprising a non-woven material having hydrophilic and hydrophobic segments; and
a coating applied to said porous, polymeric fabric.

11. The blood filter assembly of claim 10 wherein said filter medium comprises a stack of a plurality of said filter sheets.

12. A method of making a filter for removing substances from blood comprising:
forming a fabric sheet comprising polyether-ester copolymer; and
coating at least one side said fabric sheet with a coating solution comprising a copolymer of vinyl acetate and vinyl pyrrolidone.

13. The method of claim 12 wherein said coating comprises dipping said fabric in a bath of said coating solution.

14. The method of claim 13 further comprising removing excess coating solution from fabric sheet.

15. The method of claim 12 further comprising drying said coated fabric sheet at a temperature of 54°-111° C.

16. The method of claim 12 comprising forming said fabric from melt-blown fibers made of said polyether-ester copolymer.

17. The filter assembly of claim 10, wherein the porous, polymeric fabric comprises a polyester-ester copolymer.

18. The filter assembly of claim 10, wherein the porous, polymeric fabric comprises a polymeric structure of:

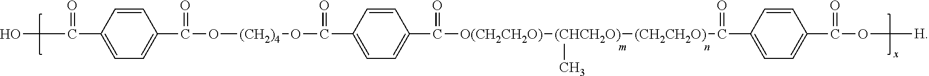

19. The filter assembly of claim 10, wherein the hydrophobic segments include repeating units derived from an alkylene glycol and at least one aromatic dicarboxylic acid or ester.

20. The filter assembly of claim 10, wherein said hydrophilic segments derived from at least one polyalkylene oxide glycol.
